(19) United States
(12) Patent Application Publication
(10) Pub. No.: US 2018/0214582 A1
(43) Pub. Date: Aug. 2, 2018

(54) METHOD FOR DIAGNOSING CARCINOMAS USING IRGD AND MAGENTIC RESONANCE TOMOGRAPHY (MRT)

(71) Applicant: Albrecht Piiper, Heidelberg (DE)

(72) Inventors: Albrecht PIIPER, Heidelberg (DE); Christian SCHMITHALS, Frankfurt am Main (DE); Thomas J. VOGL, Frankfurt am Main (DE)

(21) Appl. No.: 15/748,895

(22) PCT Filed: Aug. 1, 2016
(86) PCT No.: PCT/EP2016/068356

§ 371 Date: Jan. 30, 2018

(30) Foreign Application Priority Data
Jul. 30, 2015 (EP) 15179205.8

(51) Int. Cl. A61K 49/18; A61K 38/08; G01R 33/56; A61K 49/10; A61B 5/055; A61K 47/64

(52) U.S. Cl. CPC A61K 49/18; A61K 38/08; A61K 47/64; A61K 49/105; A61B 5/055; G01R 33/5601

(57) ABSTRACT

The present invention relates to an imaging diagnostic method, comprising a contrast agent including a contrast enhancer in a magnetic resonance imaging (MRI) assisted diagnosis of carcinoma diseases or carcinomas, in particular of hepatocellular carcinoma (HCC). The contrast agent is preferably a gadolinium compound, preferably Gd-DTPA including iRGD as a contrast enhancer, for the improved imaging of carcinomas, in particular HCC, in MRI. The invention furthermore relates to a method for the risk stratification of patients and subjects using the aforementioned diagnosis.

The present invention relates to an imaging diagnostic method, comprising a contrast agent including a contrast enhancer in a magnetic resonance imaging (MRI) assisted diagnosis of carcinoma diseases or carcinomas, in particular of hepatocellular carcinoma (HCC). The contrast agent is preferably a gadolinium compound, preferably Gd chelators including iRGD as a contrast enhancer for the improved imaging of carcinomas, in particular HCC, in the MRI. The invention furthermore relates to a method for the risk stratification of patients and subjects using the aforementioned diagnosis.

Despite the progress that has been made in recent years, the treatment of advanced-stage solid malignant neoplasms, which also include hepatocellular carcinoma (HCC), remains unsatisfactory. HCC is the fifth most common malignant neoplasm in the world, and since it is not diagnosed until at a late stage and there are no promising treatment options, it is the third most common cause of cancer-related deaths. HCC generally develops as a result of cirrhosis of the liver, which is related to chronic hepatitis. Key causes of liver cirrhosis are hepatitis B and hepatitis C virus infections, aflatoxins, and alcohol abuse.

Curative treatment approaches of HCC are liver transplant or resection. While a transplant can be carried out in BCLC stages O, A, and possibly B, tumor resection is only useful in stage 0. The remaining, more advanced tumor stages can solely be treated by local ablative procedures (LITT, TACE) or palliatively by systemic treatment (chemotherapy with sorafenib). As a result, making an early diagnosis is crucial for a successful treatment of solid malignant neoplasms. Improved monitoring of patients at high risk of developing HCC, which is to say patients suffering from liver cirrhosis, and diagnosing suspicious nodules are of central importance for a considerable improvement in the prognosis of HCC patients. Dynamic contrast magnetic resonance imaging (contrast MRI) and contrast computed tomography allow the most reliable imaging HCC diagnostics. Being supplied via the hepatic artery, HCCs exhibit a characteristic arterial inflow and venous outflow behavior during these examinations, which is utilized by the low-molecular-weight contrast agents (Magnevist®, Primovist®). Small HCCs, however, rarely exhibit the typical radiological criteria and are rather hypovascular because the supply via the portal vein is already reduced, and because arterial hypervascularization has not yet formed. Consequently, it remains difficult to make the clinically extremely significant distinction between small HCCs and regenerate and dysplastic nodules in the cirrhotic liver.

Magnetic resonance imaging (MRI) is an imaging method that is used primarily in medical diagnostics for representing the structure and function of tissues and organs in the body. Magnetic resonance imaging is based on very strong magnetic fields and alternating electromagnetic fields in the radio frequency range that are used to excite specific atomic nuclei (mostly, hydrogen nuclei/protons) in the body to move by resonance, which then induce electrical signals in a receiver circuit. Contrast agents are used to improve the representation of structures and functions of tissues/organs in the MRI. During magnetic resonance imaging, primarily gadolinium chelates are used as the contrast agent, which due to the paramagnetic property of the gadolinium atom shorten the relaxation times (T1 and T2) in the vicinity of the contrast agent and thereby result in a brighter representation of structures (higher intensity of signals).

Proceeding from this prior art, it is therefore the object of the invention to provide new options for the diagnosis, differential diagnosis, prognosis, and in particular for the early detection, of carcinomas, in particular of hepatocellular carcinoma (HCC), and for the risk stratification of such carcinoma diseases.

Above all, the technology of magnetic resonance imaging in the diagnosis of carcinoma is to be improved. Furthermore, the present invention is to offer a novel MRI contrast agent that exceeds the contrast agent presently available for the representation of the carcinoma in the MRI both in terms of specificity and sensitivity. This also allows earlier detection of small carcinomas and metastases, which so far have remained undetected in the prior art.

The above-described object is achieved by the use of iRGD as a contrast enhancer in a method for the diagnosis and/or risk stratification of carcinoma diseases or carcinomas, comprising a lanthanide compound as the contrast agent, wherein an MRI is carried out on a patient or subject.

Advantageously, iRGD allows the improved uptake and specific accumulation of a contrast agent in a carcinoma, in particular of lanthanide compounds as the contrast agent.

The invention thus relates to an imaging method for the diagnosis and/or risk stratification of carcinoma diseases, wherein by way of
a.) a contrast agent comprising a free lanthanide compound, and
b.) a contrast enhancer iRGD
c.) magnetic resonance imaging is carried out on a patient/subject.

The invention furthermore relates to a contrast agent comprising a.) a free lanthanide and b.) a contrast enhancer iRGD for use in an imaging method for the diagnosis and/or risk stratification of carcinoma diseases, wherein magnetic resonance imaging is carried out on a patient/subject.

In a further preferred embodiment of the invention, the magnetic resonance imaging is carried out multiple times, wherein, preferably, in a first magnetic resonance imaging process the
i.) contrast agent comprising a free lanthanide compound is administered/applied without a contrast enhancer iRGD, or
i.′) a contrast enhancer iRGD is administered/applied without a contrast agent comprising a free lanthanide compound, and in a delayed
ii.) second magnetic resonance imaging process the contrast agent comprising a free lanthanide compound is administered/applied with a contrast enhancer iRGD.

This allows monitoring of the achieved effect to improve the imaging process by the contrast enhancer, as well as standardization or normalization. The second MRI can preferably take place within 10 min to 6 h, in particular 1 to 24 h, in particular within several days, in particular within 4 to 24 h after the first MRI.

In particular, a corresponding injection solution may be administered, wherein a first injection solution does not comprise iRGD, but a contrast agent comprising a free lanthanide compound, and a second otherwise identical injection solution additionally comprises iRGD.

In particular, a corresponding injection solution may be administered, wherein a first injection solution does not comprise a contrast agent comprising a free lanthanide compound, but comprises iRGD, and a second otherwise identical injection solution additionally comprises a contrast agent comprising a free lanthanide compound.

The invention thus likewise relates to an imaging method for the diagnosis and/or risk stratification of carcinoma diseases, characterized in that, by way of a.) a contrast agent comprising a free lanthanide compound, or a′.) a contrast enhancer iRGD,
b.) a first magnetic resonance imaging process is carried out on a patient/subject, and with delay, by way of
c.) a contrast agent comprising a free lanthanide compound, and
d.) a contrast enhancer iRGD,
e.) a second magnetic resonance imaging process is carried out on a patient/subject.

In the context of the present invention, contrast agents that may be used are those comprising a paramagnetic substance, such as elements of the transition metals, lanthanides, and the actinides which, to the extent this is desired, may be covalently or non-covalently bound to complexing agents (chelators) or to amino acid-containing macromolecules.

Particularly preferably, elements are selected from the group consisting of Gd(III), Mn(II), Cu(II), Cr(III), Fe(II), Fe(III), Co(II), Er(II), Ni(II), Eu(III) and Dy(III).

Particularly preferred elements are Gd(III), Mn(II), Cu(II), Fe(II), Fe(III), Eu(III) and Dy(III), and in particular Mn(II) and Gd(III).

In a further very preferred embodiment, the contrast agent comprising a paramagnetic substance comprises a compound of an element selected from the group of the lanthanides. It is particularly preferred that the lanthanide compound is a gadolinium compound.

The contrast agent preferably comprises a paramagnetic substance, in particular a complexed/chelated lanthanide compound. Preferred chelators of the present invention include: acetylacetone (acac), ethylenediamine (en), 2-(2-aminoethylamino)ethanol (AEEA), diethylenetriamine (diene), iminodiacetate (ida), triethylenetetramine (triene), triaminotriethylamine, nitrilotriacetate (nta), ethylenediaminetriacetate (ted), ethylenediaminetetraacetate (edta), diethylenetriaminepentaacetate (DTPA) 1,4,7,10-tetraazacyclododecane-1,4,7,10-tetraacetate (DOTA) (also: gadoteric acid, Acidum gadotericum), oxalate (ox), tartrate (tart), citrate (cit), dimethylglyoxime (dmg), 8-hydroxyquinoline, 2,2′-bipyridine (bpy), 1,10-phenanthroline (phen), 2-[4-(2-hydroxypropyl)-7,10-bis(2-oxido-2-oxoethyl)-1,4,7,10-tetrazacyclododec-1-yl]acetate (gadoteridol, ProHance®), 2,2′,2″-(10-((2R,3S)-1,3,4-trihydroxibutane-2-yl)-1,4,7,10-tetraazacyclododecane-1,4,7-triyl)triacetate (Gadovist, Gadobutrol).

Likewise, the contrast agent according to the invention or the lanthanide compound, in particular gadolinium compound, can furthermore comprise auxiliary agents, in particular salt-forming agents, such as meglumine (also: megluminum) (N-methyl-D-glucamine) or the like, for example gadoteric acid (DOTA) in the form of gadoterate meglumine (Dotarem®). The auxiliary agent meglumine is preferred.

In a next preferred embodiment, the present invention furthermore relates to a contrast agent comprising a paramagnetic substance, in particular a lanthanide compound, wherein the gadolinium compound is a complex made up of gadolinium and a chelator, preferably wherein the chelator is selected from the group consisting of diethylenetriaminepentaacetic acid (DTPA), 1,4,7,10-tetraazacyclododecane-1,4,7,10-tetraacetic acid (DOTA) (also: gadoteric acid, Acidum gadotericum), 2-[4-(2-hydroxypropyl)-7,10-bis(2-oxido-2-oxoethyl)-1,4,7,10-tetrazacyclododec-1-yl]acetate (gadoteridol, ProHance®), or 2,2′,2″-(10-((2R,3S)-1,3,4-trihydroxibutane-2-yl)-1,4,7,10-tetraazacyclododecane-1,4,7-triyl)triacetate (Gadovist, Gadobutrol). The gadolinium compound is preferably Gd-DTPA, or derivatives of this compound, such as Gd-EOB-DTPA (Primovist®).

A “chelator” in the context of the present invention shall be understood to mean a ligand having more than one free electron pair, which can take on at least two coordination sites (binding sites) of a central atom, in particular of a gadolinium atom here. The ligands and the central atom are linked by coordinate bonds. This means that the binding electron pair is provided solely by the ligand. The entire complex made up of the central atom and the ligand is referred to as the “chelate complex.”

The essential aspect within the scope of the invention described here is that the contrast agent comprising a paramagnetic substance, this being the lanthanide compound, and preferably the gadolinium compound, is present in a free and unbound state, and in particular is not incorporated in a nanoparticle. According to the invention, a free lanthanide compound is thus particularly preferred as a contrast agent.

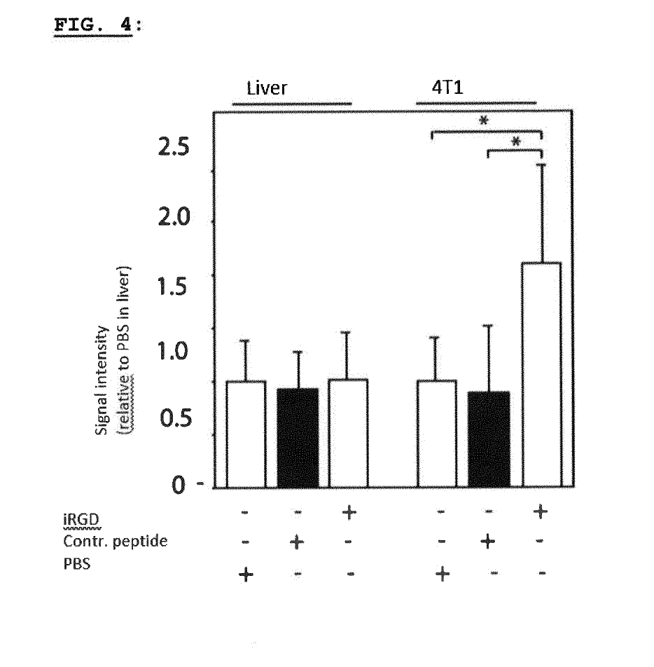

WO 2012/113733 A1 describes a contrast agent exclusively on the basis of a nanoparticle, wherein the contrast agent and the contrast enhancer are bound to a nanoparticle. However, this has not proven effective (Waralee Watcharin, 2015) since the injection of Gd-DTPA-containing human serum albumin (HSA) nanoparticles in fact causes negative contrasting of the HCCs in the livers with HCC (FIG. 4, Waralee Watcharin (supra)). It was found that the HSA nanoparticles are absorbed very rapidly by the macrophages (FIG. 7, Waralee Watcharin (supra)), so that the nanoparticles remain in the liver since the macrophage density in the liver is higher than in the malignant neoplasm.

For the purpose of a good diagnosis of carcinomas, in particular HCC, and in particular to enable a high resolution of the images in the MRI, it is preferred to use the contrast agents of the present invention in conjunction with preferably a 3-Tesla tomograph in the MRI.

According to the invention, preferred carcinomas are those that are related to a disturbance of the barrier function of the vessels and decreased lymphatic drainage in the region subject to pathological changes. This applies to most solid malignant neoplasms, in particular breast cancer, colon cancer, pancreatic cancer, stomach cancer, ovarian cancer, biliary duct cancer, prostate cancer, cervical cancer, glioblastoma, bronchial cancer, pancreatic cancer, prostate cancer, renal cell cancer, bladder cancer, brain tumors, sarcomas, hepatocellular carcinoma or tumor metastases of these cancers. Hepatocellular carcinoma (HCC), however, is particularly preferred.

In one particularly preferred embodiment, the contrast agent according to the invention furthermore comprises a tumor-homing peptide, and in particular iRGD, preferably having the sequences CRGDKGPDC (SEQ ID No.1), CRGDRGPDC (SEQ ID No.2), CRGDKGPEC (SEQ ID No.3) and CRDGRGPEC (SEQ ID No.4).

Recently it was shown in various tumor models that an intravenously applied tumor-homing peptide (iRGD) selectively improves the penetration of low and high molecular weight substances in the tumor stroma in various tumor models (Sugahara et al, 2009; 2010). iRGD contains an RGD motif which binds to integrin aᵥ, an integrin preferably expressed in the endothelial cells of tumor vessels (Ruoslahti, 2002), and a CendR motif, which binds to neuropilin-1. The binding of CendR to neuropilin-1 is necessary for the increased tumor-selective penetration effect.

The dissertation (http://thesis.library.caltech.edu/7084/45/NG_SHEUNGCHEETHOMAS_2012_CH5.pdf) likewise describes the use of Gd-DTPA and iRGD to prove that tumor vessels have slightly increased permeability. However, it is noted that this permeability that was found is not sufficiently suitable for MRI-based diagnosis.

Furthermore, WO 2011/005540 A1 describes the therapeutic use by way of iRGD for the improved penetration of cancer drugs into cancer cells.

In a further embodiment, the contrast agent according to the invention can moreover comprise agents that cause an increase in the blood pressure of the patient/subject to be diagnosed. It was possible to demonstrate, for example, that an increase in blood pressure enhances what is known as the EPR effect.

This enables improved contrasting, and thus a more specific and more sensitive diagnosis. It is thus particularly preferred that the contrast agent according to the invention comprises angiotensin II. In a further embodiment of the invention, the contrast agent can comprise a physiologically compatible aqueous medium.

In a further aspect, the object of the present invention is achieved by a method for the diagnosis and/or risk stratification of carcinomas, in particular hepatocellular carcinoma, comprising the administration of a contrast agent according to the invention, including a contrast enhancer, to a patient/subject, and a subsequent MRI scan of the patient/subject. The MRI scan is preferably carried out by way of a 3-Tesla tomograph.

The present invention can, in general, be used for acquiring images from a patient/subject by way of an MRI. The image acquisition process is carried out as follows: First, a sufficient amount of the contrast agent according to the invention, including the contrast enhancer, is administered to a patient, so as to then carry out a scan of the patient using magnetic resonance imaging. An invasive procedure is not required. In this way, images of the inner structures of the patient which are of interest, in particular of the diseased tissue, including the carcinoma, are acquired. The contrast agent according to the invention, including the contrast enhancer, is particularly helpful for the visualization of tissue, and in particular liver tissue. It may be used to acquire images of any other region of the patient/subject.

The administration of the contrast agent according to the invention, including the contrast enhancer, can be carried out in any manner best known to a person skilled in the art. This includes in particular the intravenous, parenteral, intracardiac (heart injection), oral, rectal administration, and so forth, of different formulations of the contrast agent. The required dose of the contrast agent is varied as a function of the age, the size and the weight of the patient, as well as the body region to be examined.

In a preferred embodiment, however, the contrast agent according to the invention, preferably Gd-DPTA, is applied in a dose of 45 to 65 μmol/kg, and preferably 57 μmol/kg body weight. The application is preferably carried out intravenously on a patient/subject.

In a preferred embodiment, however, the contrast enhancer iRGD according to the invention is applied in a dose of 0.1 to 12 μmol/kg, 2 to 10 μmol/kg, and preferably 4 μmol/kg body weight. The application is preferably carried out intravenously on a patient/subject.

The contrast agent including the contrast enhancer of the invention may be used alone or in combination with other diagnostic, therapeutic or other substances. “Other substances” shall in particular be understood to mean pharmaceutical adjuvants, flavor additives or dyes. For example, sucrose or a natural citrus flavor may be admixed to an orally administered formulation.

In a further embodiment, the invention relates to a kit comprising one or more injection solutions for carrying out a method according to the invention, comprising independently of one another, collectively or respectively a contrast agent, preferably Gd-DPTA and/or a contrast enhancer iRGD at a dose of 0.1 to 12 μmol/kg, 2 to 10 μmol/kg, and preferably 4 μmol/kg body weight.

For example, an injection solution comprises 300 μmol iRGD for a patient weighing 75 kg. Gd-DTPA can be administered in a customary dose for MRI.

According to the invention, the term “risk stratification” comprises the identification of patients, in particular emergency patients and at-risk patients, having a worse prognosis, for the purpose of more in-depth diagnostics and therapy/treatment of carcinoma diseases, in particular HCC, with the goal of enabling as favorable a progression of the disease as possible. Risk stratification according to the invention consequently allows effective treatment processes, which in the case of carcinoma diseases are achieved through newer drugs, such as cytostatic drugs, monoclonal antibodies and chemotherapy, for example, or are used for the treatment or therapy of carcinoma diseases.

The invention therefore likewise relates to the identification of patients who are at an increased risk of and/or have an unfavorable prognosis for carcinoma diseases, and more particularly in symptomatic and/or asymptomatic patients, in particular emergency patients, for example due to metastasization.

Particularly advantageously, reliable stratification can take place by way of the method according to the invention in particular in cases of emergency and/or intensive medicine. The method according to the invention thus enables clinical decisions that result in rapid treatment success and the prevention of deaths. Such clinical decisions likewise include advanced treatment using drugs for the treatment or therapy of carcinomas.

The invention thus likewise relates to a method for the diagnosis and/or risk stratification of patients affected by carcinoma diseases for carrying out clinical decisions, such as advanced treatment and therapy using drugs, preferably in the time-critical field of intensive medicine or emergency medicine, including the decision to hospitalize the patient.

In a further preferred embodiment, the method according to the invention thus relates to the therapy control of carcinoma diseases or carcinomas.

In a further preferred embodiment of the method according to the invention, for the diagnosis and/or risk stratification takes place for prognosis, for early detection and detection by differential diagnosis, for assessment of the severity, and for assessment of the course of the disease concomitant with the therapy.

The present invention is to be described in greater detail hereafter based on examples and figures, without thereby limiting the invention.

### EXAMPLES AND FIGURES

### 1. Peptides

Synthetic peptides iRGD (CRGDKGPDC) and the RGD control peptide (CRGDDGPKC), which have in a circular shape over a cysteine-cysteine disulfide bond between AS 1 and 9 (Sugahara et al., 2010), were acquired from GenScript USA Inc. with a purity of more than 98%.

### 2. Creation of TGFα/c-Myc Transgenic Mice and Visualization of the HCCs

Male TGFα/c-myc bitransgenic mice were created by crossing homozygous metallothionein/TGFα and albumin/c-myc single-transgenic mice in a CD13B6CBA background as described (Murakami et al., 1993; Haupenthal et al., 2012). After weaning, the animals were given ZnCl₂ by way of the drinking water to induce tumors via the expression of TGFα. Starting at an age of 20 weeks, the TGFα/c-myc animals underwent a Primovist-enhanced MRI scan in a 3T MRI Scanner (Siemens 113 Magnetom Trio, Siemens Medical Solutions) as described (Haupenthal et al., 2012; Watcharin et al., 2015; Korkusuz et al., 2013).

Mice with HCC according to the Primovist-enhanced MRI underwent the following experiments.

### 3. Creation of the HCC Nude Mice

HepG2- and Huh7 cells (ATCC and RIKEN BioResource Center) were cultivated in DMEM with 10% FBS and penicillin/streptomycin (Life Technologies). 5 million cells in 100 μL PBS were injected into the sides of NMRI Foxn1 nude mice (Harlan Laboratories B.V.). Four weeks later the mice were assigned to the test groups.

### 4. 4T1 Breast Cancer Mouse Model

4T1 cells (ATCC) were cultivated in RPMI 1640 medium with 10% FBS and penicillin/streptomycin (Life Technologies). BALB/c mice were injected with 2.5×104 4T1 cells into mammary gland no. 4 of the mouse, and the tumors were allowed to grow for two weeks.

5. Gd-DTPA-Enhanced MRI with and without iRGD

So as to determine the effect of iRGD on the Gd-DTPA-enhanced MRI in the HCC, TGFα/c mice with HCC according to a prior Gd-EOB-DTPA (Primovist)-enhanced MRI one week earlier, or nude mice with HepG2 or Huh7 tumors, were included in the experiments. The mice were anesthetized by way of intraperitoneal injection of ketamine (70 mg/kg body weight) and xylazine (10 mg/kg body weight), followed by a basal T1-weighted MRI. Directly thereafter, a Gd-DTPA-enhanced MRI was conducted (Haupenthal et al., 2012). Twelve to 24 hours later, either iRGD or the RGD control peptide was injected (100 mL each via the tail vein), followed by a basal and a Gd-DTPA-enhanced MRI (Watcharin et al., 2015). For the quantitative analysis of the MRI data, the signal intensities in user defined “regions of interest” (ROI) were used (Korkusuz et al., 2013). ROIs were placed in the liver and in the tumor tissue. The changes in the signal intensities were ascertained by subtraction of the pre-contrast value from those after the administration of Gd-DTPA. The changes in the signal intensities of the tumors and the livers due to iRGD or the RGD control peptide were indicated as multiples of the values of the Gd-DTPA MRI with prior injection of PBS.

### Description of the Results:

Co-administration of iRGD, however not of a peptide containing an RGD motif, not however a CendR motif, selectively increases the penetration of the Evans blue dye and doxorubicin in carcinomas, in particular HCC.

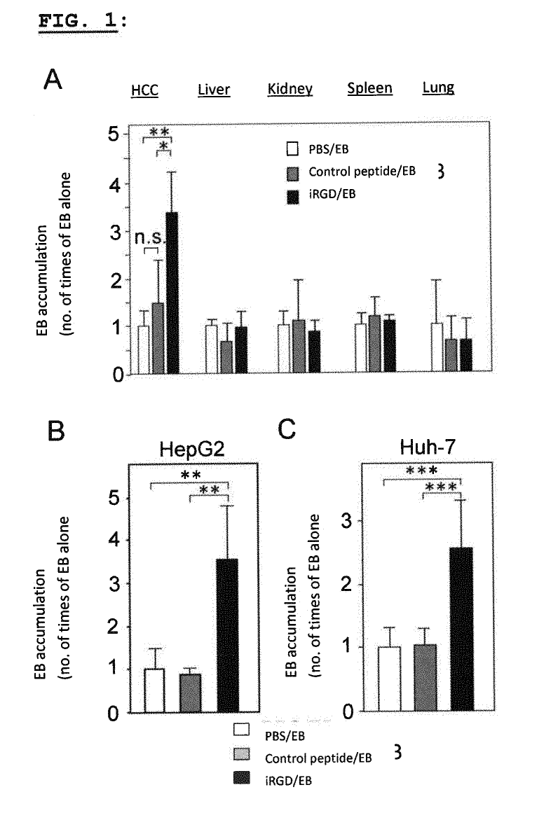

So as to examine whether intravenously applied iRGD increases the permeability of the HCC, the effect of intravenously applied iRGD on the levels of co-injected Evans blue, an albumin-binding dye, in TGFα/c-myc mice with endogeneous HCCs according to a Gd-EOB-DTPA-enhanced MRI were analyzed. For this purpose, the mice with HCC were intravenously injected with iRGD, an RGD control peptide without a CendR motif, or PBS, followed by the injection of Evans blue 15 minutes later. Another 30 minutes later, the animals were terminally perfused. The photometric quantification of the dye showed a three-fold increase in the Evans blue quantity in the tumors of iRGD-injected animals compared to HCCs of mice that had been injected with PBS (p=0.012) or the control peptide (p=0.012), while the RGD control peptide or PBS had no effect (FIG. 1A). iRGD or the RGD control peptide had no impact on the concentration of Evans blue in normal tissue (liver, kidney, spleen and lung), which supports the fact that iRGD specifically increases the permeability of the malignant tumor tissue of the HCCs in TGFα/c-myc mice for co-applied substances.

So as to examine whether the tumor-permeabilizing effect of iRGD also occurs in HCC nude mice tumor models, the effect of intravenously applied iRGD on the level of co-applied Evans blue in nude mice which had HepG2 or Huh7 xenotransplants was analyzed. iRGD effectuated an increase in the concentrations of co-applied Evans blue in HepG2-xenotransplanted and Huh-7-xenotransplanted tumors by a factor of 3.4 (p≤0.002) (in HepG2 tumors) and 2.6-fold in Huh7 tumors (p<0.001) compared to the corresponding tumors of nude mice injected with PBS and the control peptide (FIGS. 1B and C).

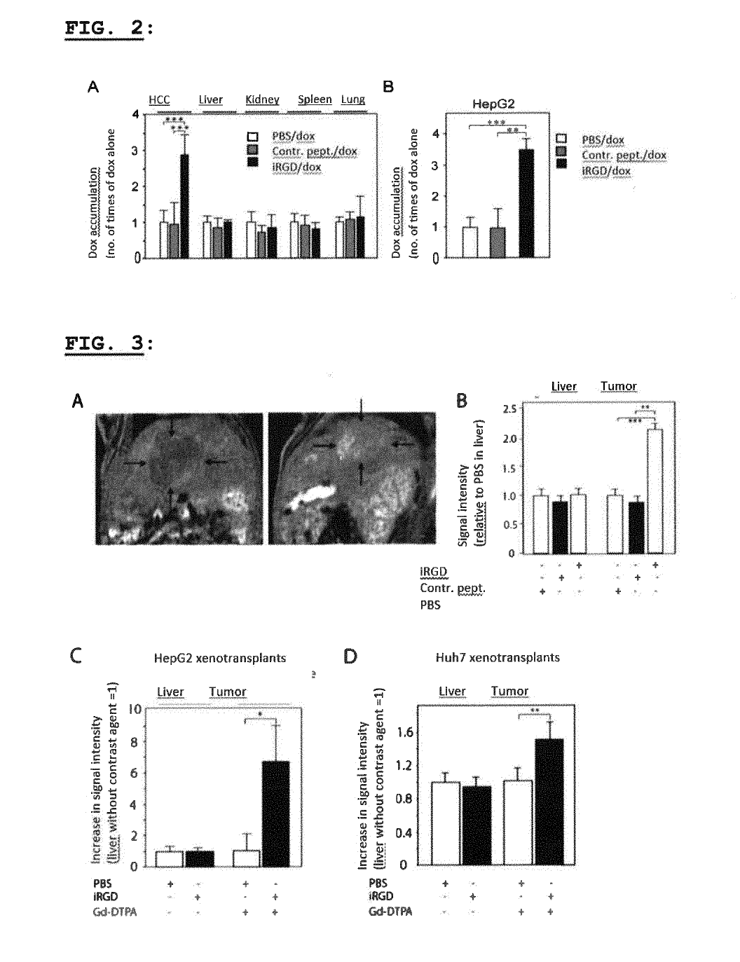

Furthermore, the effect of iRGD on the tissue concentrations of intravenously applied doxorubicin, a tumor therapeutic agent that can be detected due to the inherent fluorescence in tissue sections and tissue extracts, in the TGFα/c-myc and in the HepG2 xenotransplant HCC mouse model was analyzed. As is shown in FIG. 2, iRGD caused an increase in the doxorubicin level in the HCCs in both tumor models (p≤0.0011). iRGD had no effects on the levels of doxorubicin in the organs (FIG. 2A). The control peptide had no effects on the doxorubicin level in any of the tissues.

It was examined whether the tumor-permeabilizing effect of iRGD can already be detected by a Gd-DTPA-enhancing MRI, a widely used clinical procedure, in TGFα/c-myc mice with endogenous HCC. For this purpose, TGFα/c-myc mice with HCCs discovered one week prior according to Gd-EOB-DTPA-enhanced MRI were used. These animals received a Gd-DTPA-enhanced MRI, which showed highly negative contrasts of HCC in the liver (FIG. 3B, left). 12 to 24 hours later, the mice were administered iRGD or the RGD control peptide, followed by another Gd-DTPA-enhanced MRI. As is shown in FIG. 3A (left), the tumors of mice that had been injected PBS or the RGD control peptide were negatively contrasted. The injection of iRGD prior to the Gd-DTPA-enhanced MRI caused a substantial increase in the signal intensities of the tumors, which were now contrasting positively in the liver (FIG. 3A, right). iRGD and the RGD control peptide had no effects on the signal intensities in normal organs. The quantitative densitometric analysis of the signal intensities showed a 2-fold increase in the tumors following the injection of iRGD compared to animals who had been administered the RGD control peptide (p=0.004) or PBS (p<0.001; FIG. 3B).

Next, it was examined whether iRGD also influences the MRI signal in HCCs in HCC xenotransplant nude mice models. As is shown in FIG. 3C, iRGD caused a rise in the signal intensity of the tumors in HepG2 tumors (p=0.0311). Similar circumstances applied to Huh7 xenotransplants (p=0.0081), but the rise was less pronounced (1.5-fold; FIG. 3D) compared to the other HCC mouse models (≤2-fold). This data shows that the iRGD-induced rise in tumor permeability was able to be observed in all three different HCC mouse models by way of this non-invasive method (GD-DTPA-enhanced MRI without and with iRGD).

Furthermore, it was examined to what extent iRGD also influences the MRI signal of the tumor in the Gd-DTPA-enhanced MRI in the syngeneic 4T1 breast cancer mouse model. It was shown that iRGD resulted in a significant increase in the MRI signal in the Gd-DTPA-enhanced MRI in the 4T1 tumor (p<0.05).

The contrast enhancer iRGD thus allows a differential diagnosis so as to distinguish a benign change from a cirrhotically changed liver.

At present, the different blood supply of the liver and the tumor tissue primarily via the portal vein (liver) or the hepatic artery (HCC) for the radiological diagnostics of the HCC, which results in a delayed inflow of intravenously applied contrast agent in the liver compared to the HCC tissue, and the different distribution of anion transporters between the HCC and normal liver tissue. The latter is achieved by the distribution of the liver-specific contrast agent Primovist in the MRI.

FIG. 1:

iRGD, but not with the control peptide without a CendR motif, increased the concentration of systemically co-applied Evans blue (EB) in the HCCs in TGFα/c-myc mice and in 2 HCC transplant nude mice models.

TGFα/c-myc mice with MRI-verified HCCs (A) or mice with subcutaneous HepG2 (B) or Huh7 xenotransplants (C) were intravenously injected with 4 mmol/kg iRGD or control peptide (each in PBS), or PBS was injected alone, followed by an injection of EB 5 minutes later. The tissues were harvested after another 30 minutes. The EB accumulation in the tissues was determined by way of photometry. The data involves mean values±SD; n=4 to 5. Asterisks indicate a significant difference (* P<0.05; * P<0.01; *** P<0.001). n.s., not significant.

FIG. 2:

The co-treatment with iRGD selectively increased the concentrations of doxorubicin in HCCs of TGFα/c-myc mice and mice with HepG2 xenotransplants.

TGFα/c-myc mice with radiologically verified HCCs (A) or nude mice with subcutaneous HepG2 xenotransplants (B) were intravenously injected with 4 mmol/kg iRGD or control peptide (each in PBS), or treated with PBS alone, followed by a doxorubicin (20 mg/kg iv) injection 10 minutes later. The tissues were collected another 30 minutes later and stored, and the doxorubicin concentration was determined. The values are mean values±SD; n=5 to 6. Asterisks indicate a significant difference (**P<0.01; ***P<0.001).

FIG. 3:

iRGD resulted in a tumor-specific increase in the signal intensity in the Gd-DTPA-enhanced MRI. TGFα/c-myc mice in which HCCs had been detected one week prior by way of Gd-EOB-DTPA-enhanced MRI were anesthetized, followed by a basal T1-weighted MRI, and a Gd-DTPA-enhanced MRI directly thereafter. 12 to 24 hours later, either iRGD or RGD control peptide (con. peptide) was injected (100 μL each via the tail vein, 4 mmol/kg) into the same animals, followed by a basal MRI and a Gd-DTPA-enhanced MRI. A. Gd-DTPA-enhanced MRI of TGFα/c-myc mice with HCC (left), followed by an injection of iRGD and another Gd-DTPA-enhanced MRI 12 hours thereafter. Arrows mark the tumor.

B. quantitative analysis of the MRI data. Values are mean values±SD; n=12.

C. 12 nude mice with HepG2 xenotransplants were injected on day 1 with the control peptide, followed by a Gd-DTPA-enhanced MRI. The next day, the animals were treated with iRGD, followed by a Gd-DTPA-enhanced MRI. The values are mean values±SEM. D. 13 nude mice with Huh7 xenotransplants were subjected to the same procedure as in (C). The values are mean values±SEM. Asterisks indicate a significant difference and (* P<0.05; **P<0.01; ***P<0.001).

FIG. 4:

Effect of iRGD and the RGD control peptide on the increase in the signal intensity in the Gd-DTPA-enhanced MRI in the liver and in the 4T1 tumors in BALB/c mice. BALB/c mice were injected with 2.5×10⁴ 4T1 cells into mammary gland no. 4 of the mouse. Two weeks later, the mice were anesthetized, followed by a basal T1-weighted MRI, and a Gd-DTPA-enhanced MRI directly thereafter. 12 to 24 hours later, either iRGD or RGD control peptide (con. peptide) was injected (100 μL each via the tail vein, 4 mmol/kg) into the same animals, followed by a basal MRI and a Gd-DTPA-enhanced MRI. The data was quantitatively analyzed. Values are mean values±SD; n=6.

### LITERATURE

Sugahara K N, Teesalu T, Karmali P P, Kotamraju V R, Agemy L, Girard O M, et al. Tissue-penetrating delivery of compounds and nanoparticles into tumors. Cancer Cell 2009; 16:510-20.
Sugahara K N, Teesalu T, Karmali P P, Kotamraju V R, Agemy L, Greenwald D R, et al. Coadministration of a tumor-penetrating peptide enhances the efficacy of cancer drugs. Science 2010; 328:1031-5.
Teesalu T, Sugahara K N, Ruoslahti E. Tumor-penetrating peptides. Front Oncol 2013; 3:216.
Murakami H, Sanderson N D, Nagy P, Marino P A, Merlino G, Thorgeirsson S S. Transgenic mouse model for synergistic effects of nuclear oncogenes and growth factors in tumorigenesis: interaction of c-myc and transforming growth factor a in hepatic oncogenesis. Cancer Res 1993; 53:1719-23.
Haupenthal J, Bihrer V, Korkusuz H, Kollmar O, Schmithals C, Kriener S, et al. Reduced efficacy of the Plk1 inhibitor BI 2536 on the progression of hepatocellular carcinoma due to low intratumoral drug levels. Neoplasia 2012; 14:410-9.
Watcharin W, Schmithals C, Pleli T, Köberle V, Korkusuz H, Hübner F, et al. Detection of hepatocellular carcinoma in transgenic mice by Gd-DTPA- and rhodamine-conjugated human serum albumin nanoparticles in T1 magnetic resonance imaging. J Control Release 2015; 199:63-71.
Korkusuz H, Ulbrich K, Welzel K, Koeberle V, Watcharin W, Bahr U, et al. Transferrin-coated gadolinium nanoparticles as MRI contrast agent. Mol Imaging Biol 2013; 15:148-54.
Ruoslahti E. Specialization of tumour vasculature. Nat Rev Cancer 2002; 2:83-99.

1-15. (canceled)

16. An imaging method for the diagnosis and/or risk stratification of carcinoma diseases on a patient, characterized in that, by way of
a) a contrast agent comprising a free lanthanide compound, and
b) a contrast enhancer iRGD
c) a magnetic resonance imaging process is carried out.

17. The imaging method for the diagnosis and/or risk stratification of carcinoma diseases on a patient according to claim 16, characterized in that, by way of
a) a contrast agent comprising a free lanthanide compound, or a′) a contrast enhancer iRGD,
b) a first magnetic resonance imaging process is carried out and with delay, by way of
c) a contrast agent comprising a free lanthanide compound and
d) a contrast enhancer iRGD,
e) a second magnetic resonance imaging process is carried out.

18. The imaging method for the diagnosis and/or risk stratification of carcinoma diseases on a patient according to claim 17, wherein the second magnetic resonance imaging process takes place within 10 minutes to 6 hours, in particular 1 to 24 hours, in particular within several days, in particular within 4 to 24 hours after the first MRI.

19. The imaging method for the diagnosis and/or risk stratification of carcinoma diseases on a patient according to claim 16, characterized in that the carcinoma is selected from the group consisting of solid malignant neoplasms, breast cancer, colon cancer, pancreatic cancer, stomach cancer, ovarian cancer, biliary duct cancer, prostate cancer, cervical cancer, glioblastoma, bronchial cancer, pancreatic cancer, prostate cancer, renal cell cancer, bladder cancer, sarcomas, hepatocellular carcinoma (HCC), brain tumors, and tumor metastases of these cancers.

20. The imaging method for the diagnosis and/or risk stratification of carcinoma diseases on a patient according to claim 16, characterized in that the iRGD is selected from the group consisting of the sequences of CRGDKGPDC (SEQ II) NO: 1), CRGDRGPDC (SEQ ID NO: 2), CRGDKGPEC (SEQ ID NO: 3), and CRDGRGPEC (SEQ ID NO: 4).

21. The imaging method for the diagnosis and/or risk stratification of carcinoma diseases on a patient according to claim 16, characterized in that the free lanthanide compound is a gadolinium compound, in particular a complex made up of gadolinium and a chelator, in particular wherein the chelator is selected from the group consisting of diethylenetriaminepentaacetic acid (DTPA), 1,4,7,10-tetraazacyclododecane-1,4,7,10-tetraacetic acid (DOTA), 2-[4-(2-hydroxypropyl)-7,10-bis(2-oxido-2-oxoethyl)-1,4,7,10-tetrazacyclododec-1-yl]acetate, or 2,2′,2″-(10-((2R,3S)-1,3,4-trihydroxibutane-2-yl)-1,4,7,10-tetraazacyclododecane-1,4,7-triyl)triacetate, optionally comprising auxiliary agents, such as salt-forming agents, and in particular meglumine.

22. The method for the diagnosis and/or risk stratification according to claim 16, for the identification of patients who are at an increased risk of and/or have an unfavorable prognosis for carcinoma diseases.

23. The method for the diagnosis and/or risk stratification according to claim 16, wherein the patient is a symptomatic and/or asymptomatic patient, in particular an emergency patient.

24. The method for the diagnosis and/or risk stratification according to claim 16, for the therapy control of carcinoma diseases of a patient, in particular in intensive medicine or emergency medicine.

25. The method for the diagnosis and/or risk stratification of carcinoma diseases on a patient according to claim 16, for carrying out clinical decisions, in particular advanced treatments and therapies using drugs, in particular in intensive medicine or emergency medicine, including the decision to hospitalize the patient.

26. The method for the diagnosis and/or risk stratification of carcinoma diseases on a patient according to claim 16, for the prognosis, for early detection and detection by differential diagnosis, for assessment of the severity, and for assessment of the course of the disease concomitant with the therapy.

27. A contrast agent comprising:
a) a free lanthanide; and
b) a contrast enhancer iRGD for use in an imaging method for the diagnosis and/or risk stratification of carcinoma diseases on a patient, wherein magnetic resonance imaging is carried out.

28. The contrast agent according to claim 27, wherein, by way of
a) a contrast agent comprising a free lanthanide compound, or a′) a contrast enhancer iRGD,
b) a first magnetic resonance imaging process is carried out and with delay, by way of
c) a contrast agent comprising a free lanthanide compound, and
d) a contrast enhancer iRGD
e) a second magnetic resonance imaging process is carried out.

29. The contrast agent according to claim 27, wherein the iRGD is intravenously applied.

30. A kit comprising one or more injection solutions for carrying out the method according to claim 16, comprising independently of one another, collectively or respectively a contrast agent, in particular Gd-DPTA, and/or a contrast enhancer iRGD at a dose of 0.1 to 12 μmol/kg, 2 to 10 μmol/kg, in particular 4 μmol/kg body weight.


SEQUENCE LISTING (4 sequences)

SEQ ID NO: 1 - protein, 9 aa, Artificial sequence
  Tumorhoming-Peptid
CRGDKGPDC

SEQ ID NO: 2 - protein, 9 aa, Artificial sequence
  Tumorhoming-Peptid
CRGDRGPDC

SEQ ID NO: 3 - protein, 9 aa, Artificial sequence
  Tumorhoming-Peptide
CRGDKGPEC

SEQ ID NO: 4 - protein, 9 aa, Artificial sequence
  Tumorhoming-Peptide
CRDGRGPEC